(19) United States
(12) Patent Application Publication
(10) Pub. No.: US 2010/0063440 A1
(43) Pub. Date: Mar. 11, 2010

(54) Liquid Mixing and Injecting Tool

(76) Inventors: Ichiro Kitani, Tokyo (JP); Shigeaki Funamura, Tokyo (JP); Norifumi Fujiwara, Tokyo (JP)

Correspondence Address: TYCO HEALTHCARE GROUP LP, 15 HAMPSHIRE STREET, MANSFIELD, MA, 02048, US

(21) Appl. No.: 12/515,773

(22) PCT Filed: Jan. 25, 2008
(86) PCT No.: PCT/US08/01018

§ 371 Date: May 21, 2009

(30) Foreign Application Priority Data
Jan. 25, 2007 (JP) 2007-015249

(51) Int. Cl. A61M 37/00

(52) U.S. Cl. 604/83

(57) ABSTRACT

A liquid mixing and injecting device is provided which includes an upper branch tube, a chamber part and a rubber plug including a slit formed through the body of the rubber plug. A lid member is attached to an open end of the upper branch tube. The lid includes an opening part and a generally cylindrical member. The upper branch tube is occluded by the rubber plug. A male luer part of a syringe can be inserted into the slit to access the chamber part. A rubber plug receiving part is provided for controlling the shape of the lower end part of the rubber plug body when it is deformed to avoid forming a gap where air can accumulate within the flow path. The rubber plug is fixed by fixing an upper fixing piece of the rubber plug to the generally cylindrical member and by fixing a lower fixing piece between the opening part and the generally cylindrical member.

### TECHNICAL FIELD

The present disclosure relates to a liquid mixing and injecting tool connected to a plurality of liquid transfusion tubes for the medical use of flowing liquid medicines and the like between each of tubes.

### BACKGROUND OF THE ART

Conventionally, the predetermined liquid medicine or saline and the like are supplied to the internal body of a patient by using a plurality of liquid transfusion tubes. In such a case, between the tubes are communicated or intercepted by using a liquid mixing and injecting tool such as a medicinal plug. There is a liquid mixing and injecting tool including a plurality of branch tubes to which a rubber plug that can be penetrated by an injection needle is attached at the inner of the predetermined one of branch tubes.

This liquid mixing and injecting tool includes two branch tubes extending horizontally from a body part and an injector connecting port formed at the upper portion of the body part. Also, the injector connecting port is provided with a seal valve having an insertion part passing through from the upper side to the lower side thereof. Therefore, by inserting a twist locking injector into the insertion part of the seal valve, the twist locking injector can be communicated with the internal body of the liquid mixing and injecting tool. By this, the liquid medicine can be injected from the twist locking injector to the inner of the body of the liquid mixing and injecting tool or, conversely, the liquid medicine can be extracted from the body of the liquid mixing and injecting tool.

### SUMMARY

However, in the liquid mixing and injecting tool mentioned in above, the lower portion of the seal valve is pushed into the space of the inner of the liquid mixing and injecting tool to protrude when the twist locking injector is inserted into the insertion part of the seal valve. Therefore, air may be easily pooled around the protruded into the space of the inner of the liquid mixing and injecting tool in the seal valve. As a result, problems that an operation to eliminate the air accumulated in the liquid mixing and injecting tool or the tubes of the liquid transfusion lines is needed and that the operation is complicated can be occurred. Further, there also is the problem of the generation of bacteria in the liquid mixing and injecting tool by the air accumulated therein.

The present disclosure has been made in the light of these problems and is directed towards a liquid mixing and injecting tool in which the air is difficult to accumulate.

In furtherance thereof, the liquid mixing and injecting tool includes a mixing and injecting tool body including a chamber part, a plurality of branch tubes extending from said chamber part in different directions having at least one upper branch tube extending upwardly from the chamber part and a rubber plug attached to the upper branch tube for occluding the upper branch tube by closing a slit penetrating the inside and for communicating between the inner of the chamber part and a connecting tube by inserting the connecting tube into the slit, in which the rubber plug includes a fixing piece fixed to the upper branch tube and a rubber plug body connected to the fixing piece which is pushed into the inner lower side of the upper branch tube along the peripheral of the connecting tube by inserting the connecting tube into the slit, in which a rubber plug receiving part is provided at the inner lower side of the upper branch tube for controlling the deformation of lower end part of the rubber plug body when the rubber plug body is pushed into the inner lower side of the upper branch tube to contact to the lower end part of the rubber plug body such that the deformed lower end part of the rubber plug body has the shape that does not provide a gap in which air may be accumulated in the flow path for communicating between the connecting tube and the chamber part.

In the liquid mixing and injecting tool in accordance with the present disclosure, the lower end part positioned at the inner lower side of the upper tube of the rubber plug body is pushed to the rubber plug receiver provided at the inner lower side of the upper branch tube to contact as the rubber plug body is pushed in the inner lower side of the upper branch tube by the connecting tube when the connecting tube, for example, a male luer of a syringe is inserted into the slit of the rubber plug body to communicated the connecting tube with the chamber part. Then, almost no gap for the air accumulation within the flow path communicating between the connecting tube and the chamber part can be provided by contacting the lower end part of the rubber plug body to the rubber plug receiver.

In other words, since almost no gap other than spaces for flowing liquid within the flow path communicating between the connecting tube and the chamber part is provided, when a liquid such as liquid medicine flows from the connecting tube to the inner of the chamber part, the liquid medicine would pass through almost all portion of the spaces within the flow path communicating between the connecting tube and the chamber part. As a result, the need of a complicated operation to eliminate air in the chamber part is excluded as well as the generation of bacteria within the chamber part can be inhibited. Also, when the connecting tube is pulled out of the rubber plug body after the supply of liquid medicine or the like, the rubber plug body can be retuned to the original position by the restoring force of the fixing piece or itself and the slit is closed.

In this case, in order to inhibit the generation of the gap of air accumulated within the flowing path communicating between the connecting tube and the chamber part, for example, the shape of the rubber plug receiver is configured into a shape having the bottom part and the side parts which can be pressed by the lower end part of the rubber plug body. Further, the shape is configured such that no gap such as differential steps between the rubber plug body and the connecting tube as well as between the rubber plug body and the rubber plug receiver, respectively, when the lower end part of the rubber plug body downwardly extended along the periphery of the connecting tube is pressed to the rubber plug receiver, thereby forming a flow path in which almost no gap other than the spaces for flowing the liquid. That is, the case in which the tip of the connecting tube is projected from the lower surface of the rubber plug body and a gap can easily be generated around the projected portion or the case in which uneven surface is formed at the lower end part of the rubber plug body may be considered as a case in which the gap of air accumulated within the flow path communicating between the connecting tube and the chamber part.

Therefore, the accumulation of the air within the flow path communicating the connecting tube and the chamber part can be inhibited by minimizing the occurrence of the above cases when the connecting tube is communicating with the chamber part or when the rubber plug is occluded. Further, each of said plurality of branch tubes include a liquid flow path extended from the chamber part and comprising one upper branch tube and one or a plurality of other tubes. Moreover, any types of tools, for examples, an active plug type having a valve for communicating or blocking the predetermined branch tube or a regularly communicating type for flowing liquid medicine between branch tubes can be employed as the liquid mixing and injecting tool in accordance with the present disclosure.

For another configurational characteristic of the mixing and injecting tool of the present disclosure, a plurality of branch tubes includes an upper branch tube, one upstream branch tube generally extending horizontally towards the upstream from the chamber and a downstream branch tube extending generally horizontally towards the downstream from the chamber, in which a weir part or septum is provided for passing a liquid through the lower side portion of the upper branch tube when the liquid is flowed from the upstream branch tube to the downstream branch tube via the chamber part and a rubber receiving part is formed at both upper end sides of the weir part.

According to such a configuration, the liquid passes through the lower side of the upper branch tube (the upper side of the chamber) beyond the weir part when the liquid flows from the upstream branch tube to the downstream branch tube via the chamber part, whereby the liquid can flow all over the spaces formed at the lower side of the upper branch tube. As a result, the accumulation of the air at the lower side portion of the upper branch tube can more effectively be inhibited. That is, when the liquid flow the inner of the chamber, the liquid would pass from the space part within the chamber to all portion of the lower side portion of the upstream branch tube. Therefore, the accumulation of the air in the chamber part or the upstream branch tube can be inhibited.

Also, other configurational characteristic of the liquid mixing and injecting tool in accordance with the present disclosure is that the air is not accumulated at the lower surface of the rubber plug body can be inhibited when the liquid is flowed from the upstream branch tube to the downstream branch tube via the chamber part and passes through the lower side portion of the upper branch tube while the slit is closed to occlude the upper branch tube by the generally dome shape of the lower surface of the rubber plug body when the connecting tube is not inserted into the slit of the rubber plug. By this shape, when the slit of the rubber plug is closed and the upper branch tube is occluded against the chamber part, the flow path of the liquid flowing from the upstream branch tube to the downstream tube via the chamber part can be secured and the accumulation of the air at the lower surface of the rubber plug body can be inhibited.

Further, yet another configurational characteristic of the liquid mixing and injecting tool in accordance with the present disclosure is that the shape of the lower end part of the rubber plug body when the rubber plug body is pressed into the inner lower side of the upper branch tube by the connecting tube becomes a tapered surface downwardly expanded from the tip part of the connecting tube to rubber plug receiving part, whereby the accumulation of the air between the tip part of the connecting tube and the rubber plug receiving part can effectively be inhibited. For the tapered surface in this case, a curved surface or plane surface, or a surface formed by coupling a plurality of curved surfaces or plane surfaces may be employed.

Further configurational characteristic of the liquid mixing and injecting tool in accordance with the present disclosure is that the upper branch tube is formed into a generally cylindrical shape in which the diameter of the side portion for the chamber part is greater than that of the upper opening portion so that the rubber plug body is deformed so as to horizontally expand as lowered when it is pressed into the inner lower side of the upper branch tube.

By this configurational characteristic, the deformation amount of the rubber plug (downward extension) when the rubber plug body is pressed into the inner side of the upper branch tube as the connecting tube is inserted into the slit of the rubber plug can be reduced. Therefore, the extension of the fixing piece or the near part of the connection between the fixing piece and the rubber plug body are also small, thereby eliminating excess load applied to such portions. Thus, the rubber plug is difficult to failure. Further, this makes that the deformation into a proper shape of the lower part of the rubber plug body contacting to the rubber receiving part when the rubber plug body is pressed in to the inner lower side of the upper branch tube can readily be achieved.

Moreover, yet another configurational characteristic of the liquid mixing and injecting tool in accordance with the present disclosure is that the upper branch tube includes an opening part for connecting integrally formed with the chamber part, and a generally cylindrical shaped member attached to the opening part for connecting, and the fixing piece of the rubber plug is formed by an upper fixing piece fixed to the upper opening side of the generally cylindrical shaped member and a lower fixing piece fixed between the opening part for connecting and the generally cylindrical member.

By the above structure, it can be easy to set the rubber plug to the upper branch tube. Further, since the upper fixing piece of the rubber plug is fixed to the upper opening side of the generally cylindrical shaped member and the lower fixing piece fixed between the opening part for connecting and the generally cylindrical member, the upper fixing piece side is remarkably extended and the lower fixing piece side is deformed or slightly extended when the rubber plug body is pressed into the inner lower side of the upper branch tube by the connecting tube. Thus, the rubber plug body is returned to the original position of the upper opening side of the upper branch tube by the restoration forces of both the upper and the lower fixing pieces and the slit is occluded by the restoration force of the rubber plug body. As a result, the opening part of the upper branch tube can certainly be occluded. In this case, the rubber plug body is allowed to strongly be set because the body is supported by the upper and the lower fixing pieces.

Another configurational characteristic of the liquid mixing and injecting tool in accordance with the present disclosure is that a valve for communicating an optional branch tube in the plurality of branch tubes by shifting within the chamber is provided within the chamber. By such a structure, the communication and blocking between liquid transfusion tubes each connected to the liquid mixing and injecting tool can optionally be switched. The shift of the valve is in the direction of the rotation about an axis or the axial direction.

In the aforementioned conventional liquid mixing and injecting tool however, the seal valve body is formed in an approximately disc-shape, and a portion where the seal valve body is attached in the injection device connecting port, is formed of a concave part formed in a shape corresponding to the shape of the seal valve body. Consequently, it is difficult to allow the seal valve body to have a sufficiently large compression force for obstructing the inserting part. As a result, in cases such as when flowing the drug solutions from one side to the other side of the two branch-tubes with the seal valve body in the obstructed condition, and if the amount of the drug solutions is increased to increase the hydraulic pressure, there may be a possibility that the obstructed condition of the inserting part of the seal valve body may no longer be maintained

The present disclosure is in consideration of the foregoing situations, and discloses a liquid mixing and injecting tool having a rubber plug capable of maintaining the obstructed condition of a slit, even when the hydraulic pressure in the chamber part is increased while the slit of the rubber plug being in the obstructed condition.

In an embodiment of the liquid mixing and injecting tool according to the present disclosure, the chamber part has a tapered surface becoming gradually thinner from the chamber part side toward the upper part side of the upper part branch-tube is formed on the inner peripheral surface of the upper part branch-tube, and in addition, the portion closely contacting to the tapered surface in the rubber plug is formed so as to become gradually thinner from the portion located at the chamber part side toward the portion located at the upper part side of the upper part branch-tube, and in addition, when pressure is applied to the rubber plug attached to the upper part branch-tube from the chamber part side, the rubber plug is more strongly drawn and pressed onto the tapered surface to further secure the obstructed condition of the slit, wherein the liquid mixing and injecting tool includes the chamber part, the plural branch-tubes including the upper part branch-tube extending from the chamber part respectively to different directions, at least extending upward, and the rubber plug attached to the inner peripheral surface of the upper part branch-tube for shutting-off the upper part branch-tube by closing the slit passing through the inside as well as for allowing the communication between the inside of the chamber part and the inside of the connecting tube by inserting the connecting tube through said silt.

In the liquid mixing and injecting tool of the present disclosure, the tapered surface becomes gradually thinner from the chamber part side toward the upper part side of the upper part branch-tube is formed on the inner peripheral surface of the upper part branch-tube, and the portion closely contacting to the tapered surface in the rubber plug is formed so as to become gradually thinner from the portion located at the chamber part side toward the portion located at the upper part side of the upper part branch-tube. Consequently, the rubber plug can be attached to the inner peripheral surface of the upper part branch-tube to obstruct the slit, thereby the communication between the upper part side portion of the upper part branch-tube and the inside of the chamber part can be shut-off. And, when the hydraulic pressure inside the chamber part is increased, the rubber plug is pressed against the tapered-shape inner peripheral surface, the upper part side of which is tapered-off to the point, of the upper part branch-tube, so as to be constricted, making the obstructed condition of the slit more solid.

In this case, because of that the upper part side of the outer peripheral surface of the portion closely contacted with the tapered surface of the upper part branch-tube in the rubber plug is also formed into the tapered surface tapering-off to the point, the outer peripheral surface of the rubber plug becomes evenly and closely contacted with the inner peripheral surface of the upper part branch-tube, generating a compression force for effectively obstructing the slit at the rubber plug. Moreover, the transverse cross-section shapes of the upper part branch-tube and of the rubber plug in this case may be formed in various shapes including a round, egg, triangular and quadrangular shapes in other words, any constitution in which the tapered surface in the inner peripheral surface of the upper part branch-tube and the portion in the rubber plug closely contacted with the tapered surface are both becoming gradually thinner toward the upside than the downside, and the rubber plug is pushed against the tapered surface of the upper part branch-tube by a pressure to strongly compress the slit and allow the slit to be obstructed, may be used.

Moreover, the liquid mixing and injecting tool has an angle between the outer peripheral surface closely contacted with the tapered surface in the rubber plug in the condition not attached to the upper part branch-tube and the vertical axis is set greater than the angle between the tapered surface and the vertical axis of the upper part branch-tube. This allows, when the rubber plug receives a pressure from the chamber part side, the rubber plug to be pushed so as to be biting into the inner peripheral surface of the upper part branch-tube to strongly compress the slit. Consequently, the sealing effect of the rubber plug is further, enhanced.

A liquid mixing and injecting tool according to the present disclosure includes a chamber part, a mixing and injecting tool body having of plural branch-tubes including an upper part branch-tube extending from said chamber part respectively to different directions, at least extending upward, and a rubber plug attached to an inner peripheral surface of said upper part branch-tube for shutting-off said upper part branch-tube by closing a slit passing through the inside as well as for allowing the communication between the inside of said chamber part and the inside of said connecting tube by inserting a connecting tube through said slit, wherein a tapered surface becoming gradually thinner from said chamber part side toward the upper part side of said upper part branch-tube is formed on the inner peripheral surface of said upper part branch-tube, and in addition, a portion closely contacting to said tapered surface in said rubber plug is formed so as to become gradually thinner from a portion located at said chamber part side toward a portion located at the upper part side of said upper part branch-tube, and wherein when pressure is applied to said rubber plug attached to said upper part branch-tube from said chamber part side, said rubber plug is more strongly drawn and pressed onto said tapered surface to further secure the obstructed condition of said slit.

In addition, the liquid mixing and injecting tool may include an angle between the outer peripheral surface closely contacted with said tapered surface in said rubber plug in the condition not attached to said upper part branch-tube and the vertical axis is set greater than the angle between the tapered surface and the vertical axis of said upper part branch-tube.

In another embodiment, the liquid mixing and injecting tool includes a chamber part, a mixing and injecting body having a plurality of branch tubes extended toward different direction and at least a upper branch tube upwardly extended from the chamber part, and a rubber plug attached to the upper branch tube for occluding the upper branch tube by closing a slit passing through the rubber plug and for communicating the chamber part and a connecting tube by inserting the connecting tube into the slit, wherein, the rubber plug is formed of a fixing piece fixed to said upper branch tube and a rubber plug body attached to said fixing piece and pressed into the lower side of said upper branch tube along the outer peripheral surface of said connecting tube by inserting said connecting tube into said slit, said tool further comprising a rubber plug receiving part for controlling the deformed shape of the lower end part of said rubber plug body when said rubber plug body is pressed into the inner lower side of said upper branch tube to contact the lower end surface thereof with said rubber plug receiving part such that no gap where air is to be accumulated in a flow path communicating said connecting tube with said chamber part.

The liquid mixing and injecting tool may also include the plurality of branch tubes having said upper branch tube, an upstream branch tube generally horizontally extended upstream from said chamber part, and a downstream branch tube generally horizontally extended downstream from said chamber part, a weir part or septum is provided for passing through the lower side portion of said upper branch tube when the liquid is flowed from said upstream branch tube to said downstream branch tube through said chamber part, and said rubber receiving part is formed at both upper sides of said weir part.

The liquid mixing and injecting tool may also include the shape of the lower surface of said rubber plug body when said connecting tube is not inserted into the slit of said rubber plug is general dome shape, whereby the air is prevented from being accumulated in the lower surface of said rubber plug body when said liquid is flowed from said upstream branch tube to said downstream branch tube through said chamber part to pass through the lower side portion of said upper branch tube while said upper branch tube is occluded by closing said slit.

The liquid mixing and injecting tool can have the shape of the lower end of said rubber plug body when said rubber plug body is pressed into the inner lower side of said upper branch tube by said connecting tube is a tapered surface expanded from the tip part of said connecting tube to said rubber plug receiving part.

Alternatively, the liquid mixing and injecting tool may include said upper branch tube is formed into a generally cylindrical shape in which the diameter at the side is greater than that at the upper end opening side of said chamber part, thereby going down while it is deformed so as to horizontally expand when said rubber plug body is pressed in the inner lower side of said upper branch tube.

The liquid mixing and injecting tool may also include said upper branch tube is formed by an opening part for connecting integrally formed with said chamber part, and a generally cylindrical member attached to said opening part for connecting, and the fixing piece of said rubber plug is formed by an upper fixing piece fixed at the upper opening side of said generally cylindrical member and a lower fixing piece fixed between said opening part for connecting and said generally cylindrical member.

The liquid mixing and injecting tool may have a valve for communicating any branch tubes of said plurality of branch tube by shifting within said chamber part is placed within said chamber part.

### BRIEF DESCRIPTION OF THE DRAWINGS

Various embodiments of the presently disclosed liquid mixing and injection device are described herein with reference to the drawings, wherein:

FIG. 1 shows a top view illustrating one embodiment of the liquid mixing and injecting device in accordance with the disclosure;

FIG. 2 is a front view of the liquid mixing and injecting device shown in FIG. 1;

FIG. 3 is a side view of the liquid mixing and injecting device shown in FIG. 1;

FIG. 4 is the cross sectional view of the liquid mixing and injecting device shown in FIG. 2;

FIG. 5 is the cross sectional view of the liquid mixing and injecting device shown in FIG. 3;

FIG. 6 is a perspective view of the rubber plug of the device shown in FIG. 1;

FIG. 7 is a front view illustrating the liquid mixing and injecting device shown in FIG. 2 in which the male lure part of a syringe has been inserted into the rubber plug;

FIG. 8 is a side perspective view of the valve of the device shown in FIG. 1;

FIG. 9 is a cross sectional view of the body of the liquid mixing and injecting device shown in FIG. 1 with the valve positioned therein;

FIG. 10 is a cross sectional view of the device shown in FIG. 1 with the male lure part of a syringe inserted into the liquid mixing and injecting device;

FIG. 11 is a cross sectional view illustrating the upper branch tube, rubber plug and weir of a second embodiment of the liquid mixing and injecting device in accordance with the disclosure;

FIG. 11A is a cross sectional view illustrating the upper branch tube, rubber plug and weir of a third embodiment of the liquid mixing and injecting device in accordance with the disclosure;

FIG. 11B is a cross sectional view illustrating the upper branch tube, rubber plug and weir of a fourth embodiment of the liquid mixing and injecting device in accordance with the disclosure;

FIG. 12 is a cross sectional view of the liquid mixing and injecting device shown in FIG. 11 prior to insertion of a syringe;

FIG. 13 is a cross sectional view of the liquid mixing and injecting device shown in FIG. 11 with a syringe inserted into the rubber part;

FIG. 14 is a cross sectional view illustrating the upper branch tube, rubber plug, and weir of another embodiment of the presently disclosed liquid mixing and injecting device;

FIG. 15 is a cross sectional view of the liquid mixing and injecting device shown in FIG. 14 prior to insertion of a syringe; and

FIG. 16 is a cross sectional view of the liquid mixing and injecting device shown in FIG. 14 with a syringe inserted into the rubber part.

### DETAILED DESCRIPTION OF THE EMBODIMENTS

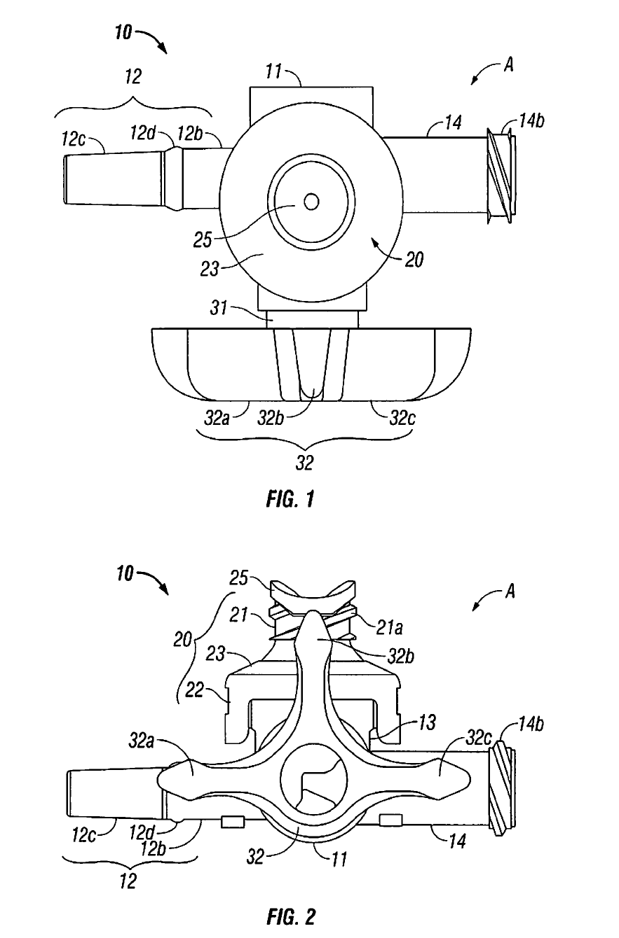

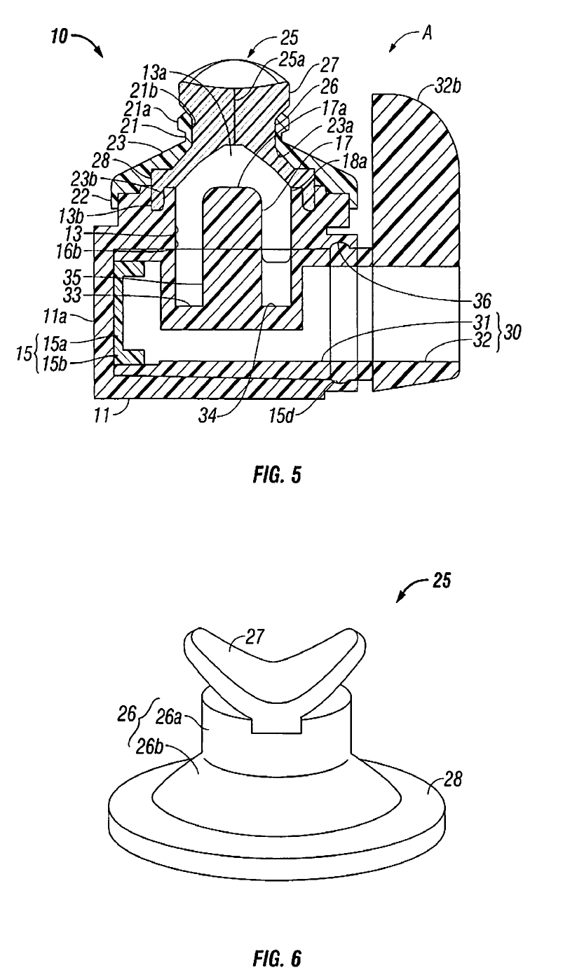

The liquid mixing and injecting tool in accordance with the present disclosure will now be explained in detail with the reference to the drawings in below. FIGS. 1 to 3 show the liquid mixing and injecting tool A in accordance with an embodiment, in which the liquid mixing and injecting tool A includes a mixing and injecting tool body 10, a lid member 20 as a generally cylindrical member of the present disclosure, a rubber plug 25 fixed within the lid member 20 (see FIGS. 4 and 5), and a valve 30 provided within the mixing and injecting tool body 10. The mixing and injecting tool body 10 includes a cylindrical chamber part 11 in which the axial length is short, three branch tubes including a downstream branch tube 12, an opening part for connecting 13 and an upstream branch tube 14 connecting in series the periphery of the chamber part 11 with the angle of 90 degrees maintained. The upper branch tube of the present disclosure is defined by the opening part for connecting 13 and the lid member 20. The rubber plug 25 is attached to the upper part side of the mixing and injecting tool main body 10 and a valve body 30 attached to the inside of the co-injection apparatus main body 10 (see FIG. 5).

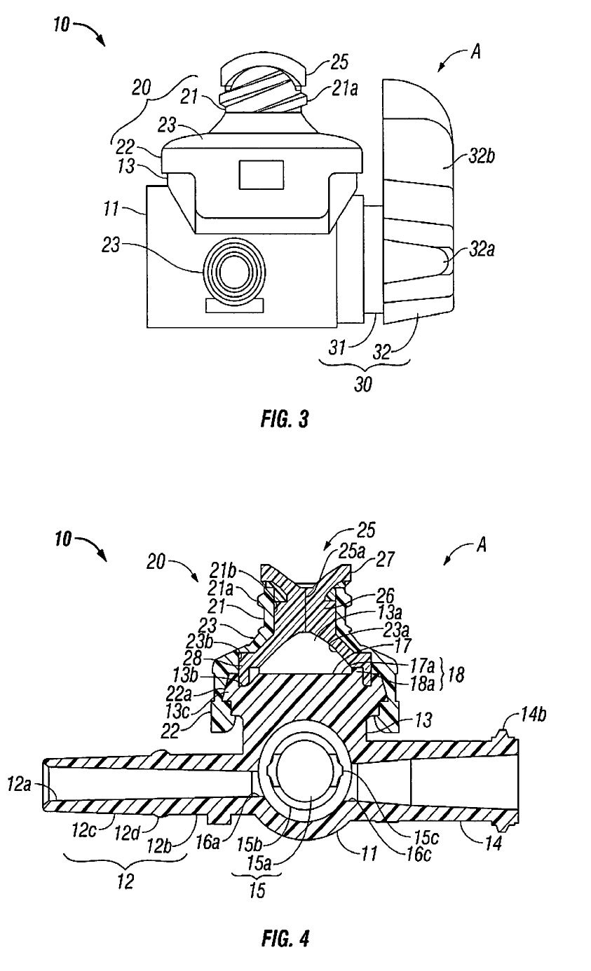

The chamber part 11 is placed with the axial direction being longitudinal direction (lateral direction in FIGS. 3 and 5), and formed into a generally cylindrical shape with the rear part is occluded. An engagement part 15 having a generally U shaped cross section is provided at the inner of the rear wall part 11a of the chamber part 11. The engagement part 15 includes a disc part 15a along the center of the interior surface of the rear wall part 11a of the chamber 11, and a ring part 15b in which axial length extended from the peripheral edge of the disc part 15a with the predetermined distance (gap) from the inner periphery is short. At both sides in the outer peripheral surface of the ring part 15 b, a pair of engaging projections 15c is formed. As shown in FIG. 4, a pair of locking projections 15c is formed at both right and left sides in the outer peripheral surface of the ring part 15b. Moreover, an engaging concave part 15d consisting of a narrow groove for inhibiting the valve body 30 from disengaging from inside the chamber part 11 is formed along the circumference at a front side position in the inner peripheral surface of the chamber part 11.

At the portions where the downstream branch tube 12, the opening for connecting 13 and the upstream branch tube 14 are connected to the peripheral wall of the chamber part 11, connecting or communication holes 16a, 16b and 16c (see FIGS. 4 to 6) are formed, respectively. The connecting hole 16b in these connecting holes 16a, 16b and 16c is formed such that the central position thereof is slightly deviated toward the front side from the central parts of other connecting holes 16a and 16c. The inner of the chamber part 11 and a flow path 12a formed within the upstream branch tube are communicated with each other through the connecting hole 16a and the inner of the chamber part 11 and a flow path 13a formed within the opening part for connecting 13 are communicated with each other through the connecting hole 16b. Further, the inner of the chamber part 11 and a flow path 14a formed within the upstream branch tube 14 are communicated with each other through the connecting hole 16c.

The communication holes 16a, 16b and 16c are formed respectively at portions where the downstream branch-tube 12, the connection opening 13 and the upstream branch-tube 14 are connected thereto in the perimeter wall part of the chamber part 11. The communication hole 16b among these communication holes 16a, 16b and 16c is formed at a location where the center part thereof is displaced slightly to the front side than the central part of the other communication holes 16a, 16c. Through the use of the communication hole 16a, inside the chamber part 11 and the flow passage 12a formed inside the downstream branch-tube 12 thereof are communicated, and through the use of the communication hole 16b, inside the chamber part 11 and the flow path 13a formed inside the connection opening 13 are communicated. Moreover, through the use of the communication hole 16c, inside the chamber part 11 and the flow passage 14a formed inside the upstream branch-tube 14 are communicated.

The downstream branch tube 12 is integrally formed with the chamber part 11 includes a basal end part 12b of the chamber part 11 side and a mal luer part 12c that is narrower than the basal end part 12b is formed at the distal end side. The male luer part 12c is formed such that the distal side portion is narrower than the basal end part 12b side portion or is tapered. At the boundary between the basal end 12b and the male luer part 12c in the peripheral surface of the upstream branch tube 12, a protrusion 12d is formed along the circumference. In particular, the protrusion part 12d is formed along the circumference at a boundary part between the base part 12b and the male luer part 12c in the outer peripheral surface of the downstream branch-tube 12.

The opening part for connecting 13 comprising the lower side portion of the upper branch tube is formed into a generally ring shape in which the diameter is greater than that of the upstream branch tube 12 and the upstream branch tube 14 and the axial length is shorter than that of tubes 12 and 14. An engaging groove 13b is formed along the periphery of the opening part for connecting 13 at almost the center of the width of the peripheral upper end portion and a pair of engaging projections 13c is formed at both sides in the outer periphery. At both sides of the inner periphery of the opening part for connecting 13, a weir part or septum 17 portioning the flow path 13a into the front and the rear portions is formed as bridged and a pair of rubber plug receiving parts 18 are formed at the portion from the inside portion of the engaging groove 13b in the upper peripheral part of the opening part for connecting 13 to the upper surface of the weir part 17.

An engaging groove 13b is formed at the approximately middle in the width direction in the upper end peripheral part along the circumference, and a pair of engagement projections 13c are formed at both right and left sides in the outer peripheral surface portion respectively.

The pair of rubber plug receiving parts 18 includes a pair of side wall parts 18a constructing wall parts of the inner periphery of the engaging groove 13b and having arc shapes from the plane view which are symmetrically formed, upwardly protruded from the top surface of the weir part 17, and both side portions of the top surface 17a of the weir part 17. Also, at the engaging protrusion 13c of the opening part for connecting 13, the lid member 20 for fixing the rubber plug 25 with the opening part for connecting 13 is provided. The lid member 20 includes a generally cylindrical body of two stages having an upper part 21 formed in a generally cylindrical shape having a short longitudinal length and a diameter slightly greater than that of the downstream branch tube 12 and the upstream branch tube 14, and a lower part 22 formed in a generally cap shape having a diameter greater than that of the opening part for connecting 13, a longitudinally long elliptic shape in the plane view, and a short axial length.

In the lid member 20, an upper part 21 is formed in an approximately cylindrical shape having a diameter set slightly larger than those of the downstream branch-tube 12 and of the upstream branch-tube 14, and having a short axial length, and is formed of a 2-stepped approximately cylindrical body, in which a lower part 22 is formed in an approximately cap shape having a diameter larger than that of the connection opening 13, having an ellipse shape having a longer anteroposterior length in the plane view, and having a short axial length. And, a sloping part 23 becoming gradually thinner from the lower part 22 side toward the upper part 21 side is formed in between the upper part 21 and the lower part 22.

The lower end parts of both side portions of the lower part 22 is extended lower than the lower end parts of both front and rear side portions and a pair of engaging recesses 22a capable of engaging with the engaging protrusions 13c of the opening part for connecting 13 are formed at the inner surface of the portion lower extended. The upper opening end part of the upper part 21 is formed into an arc shape in which the center is downwardly dimpled in the front view as shown in FIG. 2 and is formed into an arc shape in which the center is upwardly dimpled in the side view as shown in FIG. 3. A thread 21a is formed at the outer periphery of the upper part 21. The lid member 20 is attachable to the opening part for connecting 13 by engaging the engaging recesses 22a with the engaging protrusions 13c of the lower part 22. By the engagement of the lid member 20 with the opening part for connecting 13, the rubber plug 25 is fixed.

Moreover, a tapered surface 23a becoming gradually thinner from the lower part 22 side toward the upper part 21 side is formed on the inner surface of the sloping part 23, and an engaging stepped part 236 is formed at the lower part side of the tapered surface 23a. This lid member 20 is attached removably to the connection opening 13 by engaging an engaging concave part 22a of the lower part 22 to the engaging projection 13c. Moreover, the lower part side portion of the rubber plug 25 is fixed by way of the engagement between this lid member 20 and the connection opening 13.

The rubber plug 25 is formed from an elastic member such as a natural rubber, a synthetic rubber and an elastomer and includes, as shown in FIG. 6, a rubber plug body 26 having the upper part formed in a thick disc shape and the lower part formed in a generally dome shape, the upper fixing piece 27 formed at the upper end of the rubber plug body 26 and the lower fixing piece 28 having a ring shape formed around the lower peripheral edge of the rubber plug body 26. The upper fixing piece 27 is formed in a shape along the upper opening edge of the lid member 20 and includes a generally disc shape portion having a curved surface in which both sides are obliquely upwardly warped from the center the upper surface of the rubber plug body 26.

The rubber plug 26 is attached to the lid member 20 with the conditions that the rubber plug main body 26a formed at the upper part side positioned inside the upper part 21, and the dome-shaped part 26b formed at the lower part side positioned inside the sloping part 23.

Moreover, the slope angle of the outer peripheral surface of a disc-shaped part 26a (the angle between a hypothetical line (not shown) extending from the upper end of the outer peripheral surface of the disc-shaped part 26a in the perpendicular direction downward and a hypothetical line (not shown) extending up and down on the outer peripheral surface of the disc-shaped part 26a) is set greater than the slope angle of the inner peripheral surface 21b of the upper part 21 (the angle between the vertical axis of the lid member 20 and a hypothetical line (not shown) extending up and down on the inner, peripheral surface 21b). More specifically, the taper angle of the inner peripheral surface 21b of the upper part 21 is set at a more acute angle than the taper angle of the outer peripheral surface of the disc-shaped part 26a.

As shown in FIG. 5, the boundary portion between the rubber plug body 25 and the upper fixing piece 27 is formed such that the diameter thereof is greater than that of the boundary portion between the thick disc portion of the upper side and the generally dome portion of the lower side of the rubber plug body 26. Thus, the rubber plug 25 having such a structure is attached to the inside of the lid member 20 while being inhibited from detaching from the upper part 21 in the boundary portion between the rubber plug body 26 and the upper fixing piece 27. Also, as shown in FIGS. 4 and 5, the lower part fixing piece 28 of the rubber plug 25 is fixed by the opening part for connecting 13 and the lid member 20 by engaging the engaging recesses 22a of the lid member 20 with the engaging protrusions 13c of the opening part for connecting 13 while the lower fixing piece 28 is inserted into the engaging groove 13b of the opening part for connecting 13.

The upper fixing piece 27 is formed in a shape along with the shape of the upper end opening edge part of the lid member 20, and is formed from an approximately disc shaped portion having a rounded surface curved and reflexed obliquely to right and left from the middle portion in a right and left direction. Moreover, as shown in FIG. 5, the boundary portion between the disc-shaped part 26a and the upper fixing piece 27 in the rubber plug 25 is formed in a larger-diameter than that of the boundary part between the disc-shaped part 26a and the dome-shaped part 26b. And, the rubber plug 25 constituted as the foregoing is attached to the inside of the lid member 20 in the condition in which the plug is inhibited from disengaging from the upper part 21 at the boundary portion between the disc-shaped part 26a and the upper fixing piece 27.

Moreover, the lower part fixing piece 28 of the rubber plug 25, as shown in FIG. 4 and FIG. 5, is fixed by the connection opening 13 and the lid member 20 by having the engaging concave part 22a of the lid member 20 engaged to the engaging projection 13c of the connection opening 13 in the condition in which the peripheral part thereof is inserted into the engaging groove 13b of the connection opening 13. At this time, the top surface of the lower part fixing piece 28 is bent along with the engaging stepped part 23b, and the under surface of the lower part fixing piece 28 is bent along with a side wall portion 18a. Thus, the lower fixed piece 28 is interposed and bent in between the connection opening 13 and the lid member 20, thereby the lower part fixing piece 28 is more solidly secured.

In other words, the entrance of the rubber 25 into the inside of the lid member 20 is inhibited by the greater diameter of the boundary between the rubber plug body 26 and the upper fixing piece 27 as well as the exit of the rubber 25 from the upper end opening of the lid member 20 to the exterior thereof by the engagement of the opening part for connecting 13 with the lid member 20. Therefore, the rubber plug body 26 can be downwardly moved from the upper part 21 of the lid member 20 as being deformed by strongly pressed down. Conversely, when the rubber plug body 26 is moved as being deformed (extension), the upper part fixing piece 27 is stretched and the lower part fixing piece 28 is stretched or deformed.

More specifically, the rubber plug 25 is inhibited from entering into the inside of the lid member 20 by the larger-diameter portion of the boundary between the disc-shaped part 26a and the upper fixing piece 27, and also, inhibited from escaping through the upper end opening of the lid member 20 to the outside by having the lower part fixing piece 28 inserted into the engaging groove 13b and fixed by the engagement between the connection opening 13 and the lid member 20. Thereby, the rubber plug main body 26, when strongly pressed downward from the upside, can move from the upper part 21 of the lid member 20 downward while deforming, and when pressed upward from the downside, it is pushed against the inner peripheral surface 21b of the upper part 21. Moreover, when the rubber plug main body 26 moves downward (elongate) while deforming, the upper fixing piece 27 elongates, and the lower part fixing piece 28 elongates or deforms.

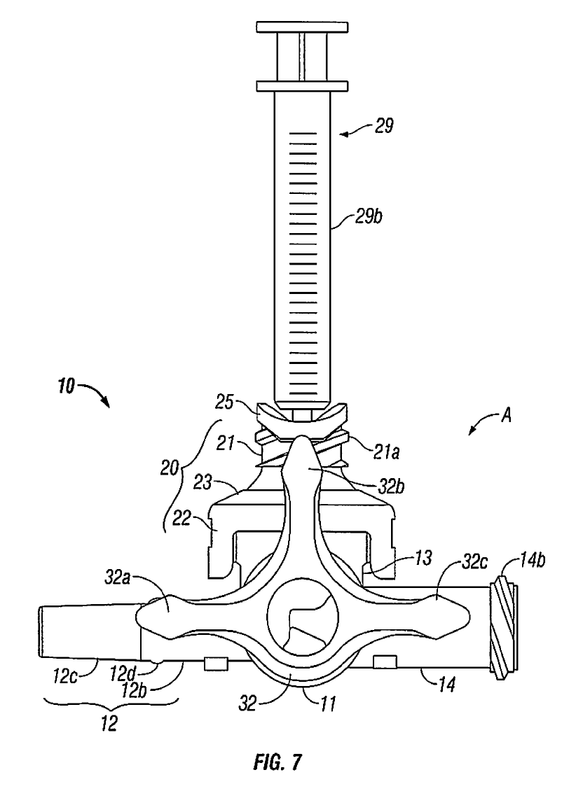

Moreover, the rubber plug body 26 of the rubber plug 25 and the upper fixing piece 27 is provided with a slit 25a for communicating the inside with the outside of the opening part for connecting 13 and for forming the flow path 13a of the opening part for connecting 13. Into the slit 25 a, for example, the male luer part 29a of the syringe 29, as shown in FIG. 7, is inserted, thereby forming a flow path for communicating a liquid medicine container 29b with the flow path 13a. Also, when the syringe 29 is not connected to the upper branch tube comprising the opening part for connecting 13 and the lid member 20, the slit 25a is in the state of occlusion by the elasticity of the rubber plug 25.

Moreover, the slit 25a for communicating between the inside and the outer side of the connection opening 13 as well as for forming a part of the flow path 13a of the connection opening 13 is provided at the disc-shaped part 26a of the rubber plug 25 and the upper fixing piece 27. This slit 25a can be maintained in the obstructed condition by the compression force that the rubber plug main body 26 receives from the inner peripheral surface of the lid member 20 upon when the rubber plug 25 is attached to the lid member 20. And, this slit 25a, into which, for example, a male luer part or connecting tube 29a of a syringe 29 shown in FIG. 7 can be inserted to form a flow passage in the male luer part 29a, which facilitates the communication between a drug solution containing part 29b of a syringe 29 and the flow path 13a.

Furthermore, when the liquid medicine container 29b is communicated with the inner of the opening part for connecting 13 by inserting the male luer part 29a into the slit 25a, the intermediate between the male luer part 29a and the peripheral surface of the slit 25a is in the contact by the elasticity of the rubber plug 25. Moreover, portions corresponding to the pair of rubber plug receiving parts 18 in the periphery of the lower surface of the rubber plug body 26 are deformed by pressing them to the rubber plug receiving part 18, respectively, to provide no gap for air accumulated at the tip side of the male luer part 29a of the syringe 29.

Moreover, in the event that the male luer part 29a is inserted into the slit 25a to allow communication between the drug solution containing part 29b of the syringe 29 and the inside of the connection opening 13, the male luer part 29a and the peripheral surface of the slit 25a are closely attached one another by the elasticity of the rubber plug 25.

That is to say, this rubber plug body 26 is previously configured such that when the body 26 is pressed down to within the opening part for connecting 13 by the male luer part 29a and the male luer part 29a is inserted into the slit 25a, the lower surface of the body 26 does not provide gaps such as differential steps as pressing to the rubber plug receiving part 18. Conversely, when the male luer part 29a is not inserted into the slit 25a, the lower surface of the rubber plug body 26 is a smooth dome like curved surface, thereby inhibiting air accumulated at the flow of liquid such as a liquid medicine on the lower surface of the rubber plug body 26.

The under surface of the dome-shaped part 26b thereof is pre-formed so as to deform to a shape which inhibit the generation of gaps such as a step or bump when pushed against the rubber plug holding part 18. Moreover, when the male luer part 29a is not inserted in the slit 25a, the under surface of the dome-shaped part 26b becomes a smooth dome-shaped curved surface so that air cannot be easily accumulated when a liquid such as a drug solution flowing under surface side of the dome-shaped part 26b. In addition, when a pressure is applied onto the rubber plug main body 26 upward from the downside due to the liquid flowing under surface side of the rubber plug main body 26, the disc-shaped part 26a is pushed against the inner peripheral surface 21b of the upper part 21 to maintain the obstructed condition of the slit 25a more securely.

The upstream branch tube 14 is integrally formed with the chamber part 11 and provided with a flow path 14a having a tapered hole formed therein. The flow path 14 a is communicated with a communicating hole 16c and has a tapered shape at the side portion of the communicating hole 16c such that the diameter of the side portion of the communicating hole 16c of the flow path 14 is less as close to the communicating hole 16c and is great as remote from thereof. Around the outer peripheral surface of the opening part of the upstream branch tube 14, a thread part 14b for connecting is formed.

This flow passage 12a is communicated with the communication hole 16c, and the communication hole 16c side portion is formed in a tapered shape having a diameter becoming smaller toward the communication hole 16c and becoming larger away form the communication hole 16c. Moreover, the upstream portion (the right hand portion in FIG. 4) of the flow passage 14a is formed in a tapered shape having a diameter becoming gradually larger toward the openings of the upstream branch-tube 14. The opening side portion of this upstream branch-tube 14 forms a female luer part for connecting the male luer part connected to the other tube or the like. And, the connecting thread part 14b is formed on the outer peripheral surface of the opening of the upstream branch-tube 14.

The valve 30 includes a generally cylindrical valve body 31 and an operation part 32 connected to the front end part of the valve body 31. The valve body 31 is provided within the chamber part 11 while the tip end of the body 31 is inserted between the inner peripheral surface of the chamber part 11 and the ring part 15b, thereby rotating about the axis of the chamber part 11 by operating the operation part 32. Also, to the tip part of the valve body 31 in the inner peripheral surface, a plurality of engaging recesses (not shown) capable of being engaged with a pair of engaging protrusions 15 c formed on the outer peripheral surface of the ring part 15b are formed with certain intervals in the direction of the circumference, whereby the valve 30 can be stopped at the predetermined three points.

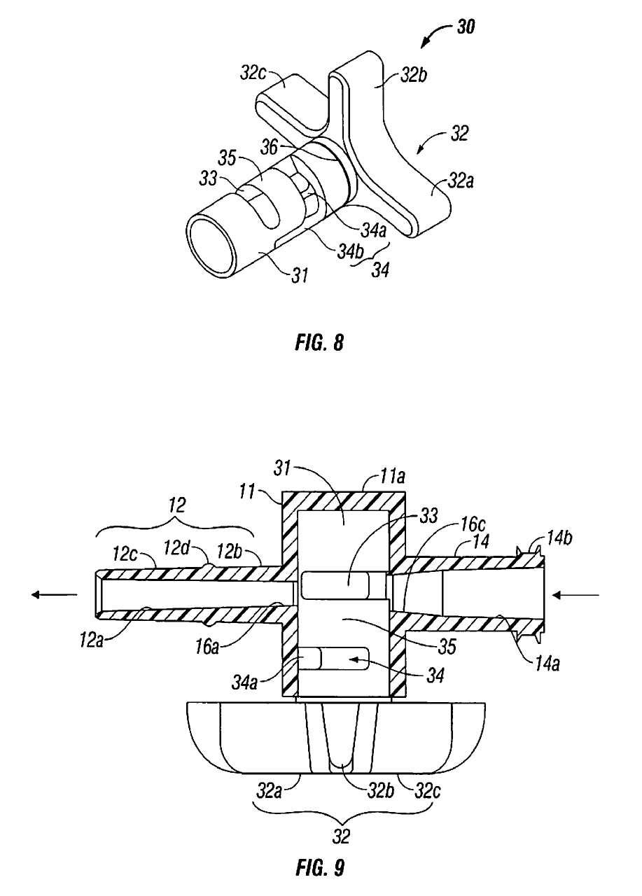

Therefore, between the communicating holes 16a, 16b and 16c of the chamber part 11 can be communicated with each other or blocked. As shown in FIG. 8, two grooves 33, 34 are formed in series in the axial direction on the outer peripheral surface. The groove 33 is formed by a notched groove extended over almost the half cycle along the circumference in the area positioned slightly posterior side apart from the center of the axis (the left side in FIG. 8) in the outer peripheral surface. The groove 34 is formed by a notched groove of generally L shape comprising a circumference groove 34a extended along the outer periphery surface of the valve body 31 parallel to the groove 33 in the area positioned slightly fore side apart from the center of the axis in the outer peripheral surface of the valve body 31, and an axial groove 34b angled at the one end of the circumference groove 34a and extended toward the rear side in the axial direction from that end.

Moreover, an engaging protruded ridge 36 engaged with the engaging concave part 15d of the chamber part 11 is formed at the operating part 32 side in the outer peripheral surface of the valve main body 31 along the circumference, and the valve body 30 is inhibited from disengaging from the chamber part 11 by the engagement of this engaging protruded ridge 36 with the engaging concave part 15d.

Also, the axial groove 34b of the groove 34 is placed at a position where the predetermined space from one end of the groove 33 is maintained, while the other end of the circumference groove 34a of the groove 34 is placed at a position at one side departed from the other end of the groove 33 along the circumference direction (fore side in FIG. 8). Further, the lengths of the grooves 33 and 34 along the circumferential direction of the valve body 31 are the same and almost a half of the circumference of the valve body 31 and the space between the groove 33 and the circumference groove 34a of the groove 34 is almost the same as the total widths of the groove 33 and the circumference groove 34a of the groove 34. Between the groove 33 and the circumference groove 34a of the groove 34, a partitioning wall 35 along the outer peripheral surface of the valve body 31 is formed.

The groove 33 is formed of a notched groove formed over an approximately half-perimeter along the circumference at a portion slightly back side (left side in FIG. 8) from the axial center in the outer peripheral surface of the valve main body 31, Moreover, the groove 34 is formed of an approximately L-shaped notched grooves consisting of a circumferential groove 34a formed along the outer peripheral surface of the valve main body .31 in parallel to the groove 33 at a portion slightly to the front side from the axial center in the outer peripheral surface of the valve main body 31, and an axial groove 34b bent from one side of end parts of the circumferential groove 34a and extending toward the back side in the axial direction.

The axial groove 34b of the groove 34 is provided at a position spaced from one side of end parts of the groove 33, and the other side of end parts of the circumferential groove .34a of the groove 34 is positioned at one side more along the circumferential direction than the other side of end parts of the groove 33 (near side in FIG. 8). Moreover, both lengths of the groove 33 and of the groove 34 along the circumferential direction of the valve main body 31 are together determined equally with the approximately half-perimeter of the circumference, and the distance between the groove 33 and the circumferential groove 34a of the groove 34 is nearly the same as the total of the width of the groove 33 and the width of the circumferential groove 34a of the groove 34. And, a partitioning wail part 35 is formed between the groove 33 and the circumferential groove 34a of the groove 34 along the outer peripheral surface of the valve main body 31.

When the valve body 31 is placed within the chamber part 11, the portion where the groove 33 and the axial groove 34b of the groove 34 are formed is aligned with the communicating holes 16a and 16c in the outer periphery surface and the other portion where the circumference groove 34a of the groove 34 is formed is opposed to the front side portion in the inner peripheral surface of the chamber part 11. Also, the outer peripheral surface of the partition wall 35 is opposed to the communicating hole 16b at the area slightly fore side from the center in the inner peripheral surface of the chamber part 11. Therefore, as shown in FIG. 9, when the valve body 31 is positioned so that the partition wall 35 towards the upper side, the groove 33 is opposed to the communicating hole 16c to communicate the inner of the chamber part 11 with the upstream branch tube 14 through the groove 33. The rear side of the axial groove 34b of the groove 34 is opposed to the communicating hole 16a to communicating the inner of the chamber part 11 with the downstream branch tube 12 trough the groove 34.

In this time, the weir part 17 is positioned at the upper of the partition wall 35 and the outer peripheral surface of the partition wall 35 is contacted with the lower surface of the weir part 17 almost stuck with each other. Since the space forming the flow path 13a is positioned at the upper of the weir part 17, the groove 33 is communicated with the groove 34 through the flow path 13a. Therefore, in this state, a liquid medicine and the like can flow from the upstream branch tube 14 to the downstream branch tube 12 through the chamber part 11 and the opening part for connecting 13. In this case, the liquid medicine and the like flowing from the upstream branch tube 14 into the groove 33 flows to the groove 34 over the weir part 17. Thus, the liquid medicine and the like can be past through the upper flow path 13a of the chamber part 11, thereby inhibiting air or so from being accumulated within the chamber part or the flow path 13a.

Since that the open space part constituting the flow path 13a is positioned at the upside of the weir part 17, the groove 33 and the groove 34 are communicated through the use of the flow path 13a. Hence, in this condition, drug solutions or the likes can be flowing from the upstream branch-tube 14 to the downstream branch-tube 12 through the use of the chamber part 11 and the connection opening 13. In this case, the drug solutions flowing from the upstream branch-tube 14 into the inside of the groove 33, flow over the weir part 17 and continue to flow into the groove 34. Consequently, the drug solutions or the likes pass through inside the flow path 13a of the upside of the chamber part 11, thereby the accumulations of air or the like in the chamber part 11 and/or in the flow path 13a can be suppressed.

As the valve 30 is rotated in one direction from that state to oppose the groove 33 to the communicating hole 16a as well as the outer peripheral surface of the valve body 31 to the communicating hole 16c, the chamber part 11 and the downstream branch tube 12 are communicated with each other and the pathway between the chamber part 11 and the upstream branch tube 14 is occluded. on the other hand, as the valve 30 is rotated in the other direction from the state as shown in FIG. 9 to maintain the position where the groove 33 is opposed to the communicating hole 16c and to oppose the outer peripheral surface of the valve body 31 to the communicating hole 16a, the pathway between the chamber part 11 and the downstream branch tube 12 is occluded and the chamber part 11 is communicated with the upstream branch tube 14.

In this way, by rotating the valve 30, both the downstream branch tube 12 and the upstream branch tube 14 can be communicating with the chamber part 11 or, or one of the tubes can be communicated with the chamber part 11. The operation part 32 includes three operational piece 32a, 32b and 32c, each being formed so as to correspond to the downstream branch tube 12, the opening part for connecting 13 (lid member 20) and the upstream branch tube 14, respectively, with the angle of 90 degrees kept, when the operational piece 32b is aligned with the downstream branch tube 12, the flow paths 12a and 13a are communicated with each other, while when the operational piece 32b is aligned with the upstream branch tube 14, the flow paths 13a and 14a are communicated with each other. In this time, the engaging protrusion 16 c is engaged with the engaging recess to stop the operational part 32 at the position as far as an external force is applied thereto.

When the operating piece 32b is positioned to the position of the lid member 20, all of the flow passages 12a, 13a and 14a are communicated, and when the operating piece 32b is positioned to the position of the downstream branch-tube 12, the flow passages 12a, 13a are communicated, and when the operating piece 32b is positioned to the position of the upstream branch-tube 14, the flow passages 13a, 14a are communicated. Moreover, at that time, a locking concave part is engaged to the locking projections 15c and the operating part 32 remains at that position unless otherwise an external force is applied.

In this configuration, with the communication between the inside of the chamber part 11 and the upstream branch-tube 14 shut-off, in the event that predetermined drug solutions or the likes are injected from the upstream branch-tube 14 to the downstream branch-tube 12 to supply the drug solutions into a patient's (not shown) body, firstly a rear end part of an infusion tube (not shown) having an indwelling needle for puncturing and indwelling is connected to the downstream branch-tube 12. Then, the male luer part provided at the tip part of the infusion tube extending from a container or the like containing the drug solution to be supplied to the patient is connected to the upstream branch-tube 14. Then, after the drug solution is passed through inside the infusion line including the chamber part 11 to release all the air inside the infusion line, the indwelling needle is punctured into the patient's body and indwelled within, and then the drug solutions in the container or the like is pumped toward the patient, thereby the supply of the drug solution into the patient is carried out.

To the liquid mixing and injecting tool A configured in this way, the syringe 29 is detachably attached and the inside of the liquid medicine container 29b is communicated with the flow path 13a of the opening part for connecting 13 by inserting the mal luer part 29a of the syringe 29 into the slit 25a of the rubber plug 25 as shown in FIG. 10. The male luer part 29a expands the slit 25a while being pressed onto the inner peripheral surface of the rubber plug body 26 to deform itself when the male luer part 29a is inserted into the slit 25a of the rubber plug 25. In this time, the rubber plug body 26 and the upper fixing piece 27 are downwardly stretched while being deformed. Therefore, the rubber plug body 25 is pressed down to the lid member 20 by the pressure force of the male luer part 29a to downwardly stretch while contacting with the inner peripheral surface of the lid member 20 and the outer peripheral surface of the male luer part 29a.

The rubber plug main body 26 and the upper fixing piece 27 extend (elongate) downward while deforming. Thereby, the rubber plug main body 26 is pressed against the lid member 20 by the pressing force of the male luer part 29a, extending downward while being closely attached to the inner peripheral surface of the lid member 20 and the outer peripheral surface of the male luer part 29a. And, when the male luer part 29a is inserted into the slit 25a to allow the drug solution containing part 29b to communicate with the flow path 13a, the lower end part of the rubber plug main body 26 is pressed against the rubber plug holding part 18, no gap is generated at the tip side portion of the male luer part 29a where air tends to accumulate.

In this case, the drug solution flows from the upstream branch-tube 14 and passes through inside the chamber part 11, and then flows toward the downstream branch-tube 12 side. At that time, the slit 25a of the rubber plug main body 26 is obstructed to inhibit the drug solution from leaking out of the rubber plug 25. Moreover, in the event that the amount of the drug solution is increased causing the hydraulic pressure inside the chamber part 11 to increase, the rubber plug main body 26 maintains the obstructed condition of the slit 25a more securely through the use of the hydraulic pressure of the drug solution, allowing the drug solution to passing through inside the chamber part 11 in an appropriate condition. Moreover, in the event that 2 kinds of the drug solutions are supplied into the patient (not shown) body, from the aforementioned condition, in the condition in which the other drug solutions is aspirated inside the drug solution containing part 29b of a syringe 29, the male luer part 29a of the syringe is made to pass completely through the slit 25a of the rubber plug 25. At this time, the male luer part 29a presses the inner peripheral surface of the rubber plug main body 26 against the lid member 20 to widen the slit 25a while making the rubber plug main body 26 deformed.

Then, when the liquid medicine container 29b is communicated with the flow path 13a by the insertion of the male luer part 29a into the slit 25a, the lower end of the rubber plug body 26 is pressed down to the rubber plug receiving part 18, thereby inhibiting any gap such as differential steps where air is easily accumulated from being provided in the tip side portion of the male luer part 29a. Therefore, as shown in FIG. 7, the liquid medicine and the like can be flowed from the upstream branch tube 14 to the downstream branch tube 12 by communicating the upstream branch tube 14 with the downstream branch tube 12 and another liquid medicine and the like from the syringe 29 can be mixed with the liquid medicine. Another liquid medicine and the like can be flowed from the syringe 29 to the downstream branch tube 12 while the pathway between the chamber part 11 and the upstream branch tube 14 is occluded.

In this configuration, when two kinds of liquid medicines are supplied into the patient's body (not shown), first, the rear end portion of a liquid transfusion tube (not shown) to which an indwelling needle for the punctuation and indwelling it to the patient is connected to the downstream branch tube 12. Secondary, the male luer part provided at the tip portion of the liquid transfusion tube extended from a container for containing one liquid medicine to be supplied to the patient is connected to the upstream branch tube 14. Then, the male luer part 29a is past through the slit 25a of the rubber plug 25 while the other liquid medicine is suctioned into the liquid medicine container 29b of the syringe 29.

Next, the liquid medicine us allowed to enter the liquid transfusion line including the chamber part 11 to discharge the air in the liquid transfusion line to the exterior, and the liquid medicine is delivered to the patient while the indwelling needle is punctured and indwelled in the body of the patient to supply the liquid medicine to the patient. And the liquid medicine in the liquid medicine container 29b of the syringe 29 is also properly injected into the chamber part 11 through the flow path 13a. In this case, the lower end portion of the rubber plug body 26 is pressed down to the rubber plug receiving part 18 to form no gap where the air is easily accumulated, while the tight junctions respective between the rubber plug receiving part 18 and the rubber plug body 26 and between the male luer part 29a and the rubber plug body 26 are achieved.

When the supply of the drug solution from the syringe 29 is completed and the male luer part 29a is pulled out from the slit 25a, the rubber plug main body 26 is released from the pressing force exerted by the male luer part 29a, retuning to the original condition by its own restoring force. Moreover, according to this liquid co-injection apparatus-A, the inside of the lid member 20 is obstructed by the rubber plug 25, thereby possible propagation of bacteria due to air entering inside the chamber part 11 can also be suppressed.

Thus, in the liquid mixing and injection tool A, the inner peripheral surface 21b of the upper part 21 in the lid member 20 is formed in a tapered surface becoming gradually thinner from the upper part side down to the lower part side, and the outer peripheral surface of the disc-shaped part 26a closely contacted with the inner peripheral surface 21b in the rubber plug 25 is formed in a tapered surface becoming gradually thinner from the lower part side up to the upper part side. The rubber plug 25 can be inserted into the inner peripheral surface of the lid member 20 to obstruct the slit 25a, thereby the communication between the upper part side portion of the lid member 20 and the inside of the chamber part 11 can be shut-off While maintaining the condition, when the hydraulic pressure of the drug solution passing through inside the chamber part 11 is increased, the disc-shaped part 26a of the rubber plug 25 is pressed against the inner peripheral surface 21b of the upper part 21, the upper part side of which is tapered-off to the point, and the rubber plug 25 makes the obstructed condition of the slit 25a more solid.

Therefore, it can be inhibited to mix air with the liquid medicines to be supplied to the patient. After the completion of the supply of the liquid medicine from the syringe 29, when the male luer part 29a is pulled out of the slit 25a, the rubber plug body 26 is released from the pressure force applied by the male luer part 29a and restored to the status shown in FIGS. 4 and 5 by its restoring force. Further, by the liquid mixing and injecting tool A, it can be inhibited that air is entered into the chamber part 11 and bacteria is propagated since the rubber plug 25 is occluded by the lid member 20.

At this conjuncture, because that the disc-shaped part 26a of the rubber plug 25 is also tapered-off to the point at the upper part side, the outer peripheral surface of the rubber plug 25 is evenly contacted with the inner peripheral surface of the lid member 20 to effectively generate a compression force at the rubber plug 25. In addition, because that the taper angle of the inner peripheral surface 21b is set at a more acute angle than the taper angle of the outer peripheral surface of the disc-shaped part 26a, when the rubber plug 25 receives a pressure from the drug solution inside the chamber part 11, the rubber plug main body 26 is pushed so as to be biting into the inner peripheral surface 21b of the lid member 20, compressing the slit 25a more strongly. Consequently, the sealing effect of the rubber plug 25 is further enhanced.

In this way, in the liquid mixing and injecting tool A of the embodiment in accordance with the present disclosure, as the rubber plug body 26 is pressed into the inner of the opening part for connecting 13 by the male luer part 29a, the lower end portion of the rubber plug body 26 is pressed down and contacted with the rubber plug receiving part 18 provided within the opening part for connecting 13 upon the male luer part 29a of the syringe 29 is inserted into the slit 25a of the rubber plug body 26 to communicate the syringe 29 with the chamber part 11. And, by the contact of the lower end portion of the rubber plug body 26 with the rubber plug receiving part 18, no gap where air to be accumulated is formed within the flow path 13a for communicating the male luer part 29a with the chamber part 11.

Incidentally, in the liquid mixing and injecting tool A, the slope angle of the outer peripheral surface of the dome-shaped part 26b (the angle between a hypothetical line (not shown) extending from the outer peripheral surface of the disc-shaped part 26a in the perpendicular direction downward and a hypothetical line (not shown) extending up and down on the outer peripheral surface of the dome-shaped part 26b) can be set greater than the slope angle of the tapered surface 23a of the sloping part 21 (the angle between the vertical axis of the lid member 20 and a hypothetical line (not shown) extending up and down on the tapered surface 23a). This allows the sealing effect when the rubber plug 25 receiving a pressure from the drug solution inside the chamber part 11 to be further enhanced.

As a result, the need to complicated operation to remove out the air in the chamber part 11 can be eliminated and the generation of bacteria in the chamber part 11 can also be inhibited. Furthermore, the rubber plug receiving part 18 is provided at the upper surface of the weir part 17 provided within the opening part for connecting 13 to flow the liquid medicine flowing from the upstream branch tube 14 to the downstream branch tube 12 through the chamber part 11 over the weir part 17, thereby more effectively inhibiting the air from being accumulated within the opening part for connecting 13. Moreover, since the shape of the lower surface of the rubber plug body 26 at the occlusion is a dome like shape, the flow path of the liquid from the upstream branch tube 14 to the downstream branch tube 12 through the chamber part 11 can be secured and the accumulation of the air on the lower surface of the rubber plug body 26 can be inhibited when the rubber plug body 26 is occluded.

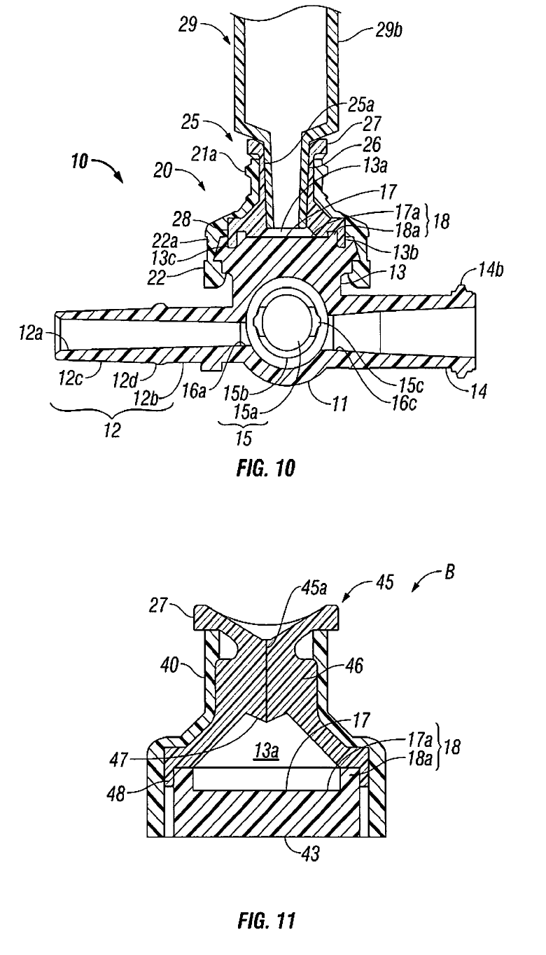

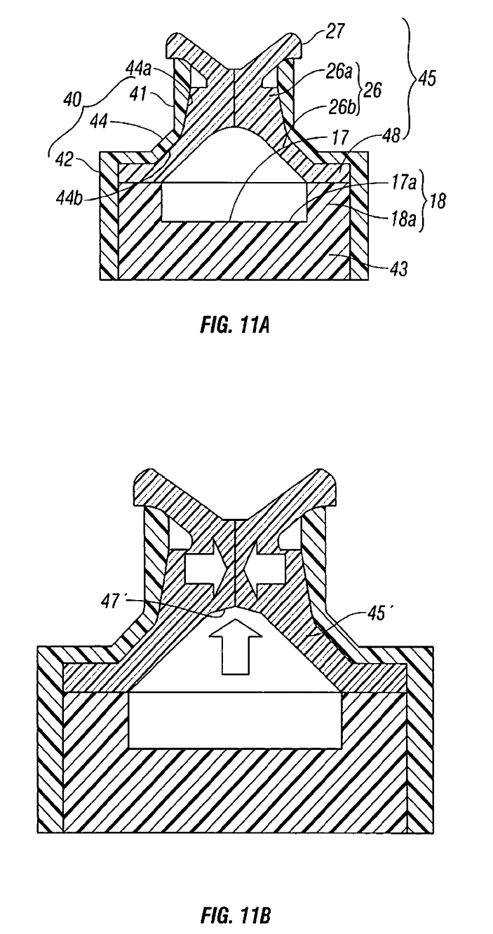

FIG. 11A shows a substantial part of a liquid co-injection apparatus according to a modified Example of the present disclosure. In this liquid co-injection apparatus, a engaging groove for fixing a lower fixed piece 48 of a rubber plug 45 is not formed at a connection opening 43, the lower fixed piece 48 of the rubber plug 45 is fixed in the condition interposed between the inner peripheral surface of a lid member 40 and the upper end surface of the connection opening 43. Moreover, the inner peripheral surface of a sloping part 44 located in between the upper part 41 and a lower part 42 of the lid member 40 is not formed in a tapered surface consisting of a smooth curved surface, but formed of 2-step tapered surfaces 44a, 44b. The configurations of other portions in this liquid co-injection apparatus are the same as those of the aforementioned liquid mixing and injecting tool A. Thus, the identical notations are assigned to the identical parts and the detailed descriptions are omitted. With the use of the liquid mixing and injecting tool, functions and effects similar to those of the liquid mixing and injecting tool may also be obtained.

Also, the diameter of the lower side portion is greater than that of the upper end opening side portion of the lid member 20 attached to the rubber plug 25, whereby the rubber plug body 26 goes down while being deformed so as to expand in the horizontal direction as pressed down into the inner lower side of the lid member 20. Therefore, the downward stretch of the rubber plug 25 can be reduced to avoid receiving excess forth. This leads the rubber plug 25 difficult to be failed. Further, since the upper fixing piece 27 of the rubber plug 25 is fixed to the upper end opening portion of the lid member 20 and the lower fixing piece 28 is fixed to the connecting portion between the opening part for connecting 13 and the lid member 20, the rubber plug 25 can strongly be set.

Moreover, a liquid mixing and injecting tool according to the present disclosure is not limited to the aforementioned embodiments and may be arbitrarily modified and implemented accordingly within the scope of the present disclosure. For example, a stopcock type apparatus having the valve body 30 is used as a liquid co-injection apparatus in each of the aforementioned embodiments, however, a liquid co-injection apparatus according to the present disclosure may be constituted without the valve body, wherein a drug solution or the like may be made to flow from an upstream branch-tube through to a downstream branch-tube, which is constantly in communication with the upstream branch-tube, and in addition the other drug solution or the like may be made to flow from the upper part branch-tube through to the chamber part. Moreover, a liquid mixing and injecting tool in which branch-tubes are formed of only the upper part branch-tube and the downstream branch-tube without the upstream branch-tube may also be used. In addition, the shapes of the inner peripheral surfaces of the lid members 20, 40, and the shapes of the outer peripheral surfaces of the rubber plugs 25, 45 may also be arbitrarily altered.

FIGS. 11 to 13 show the liquid mixing and injecting tool B of the second embodiment in accordance with the present disclosure in which a syringe 49 is connected to the liquid mixing and injecting tool B. In the liquid mixing and injecting tool B, the shape of the lower surface of a lid member 40 is formed into a generally conical shape with an upwardly concaved portion when a slit 45a in a rubber plug body 46 of a rubber plug 45 attached within the lid member 40 is occluded and a generally concave small protrusion 47 downwardly projected is formed at the mid part (upper portion) thereof. In an alternate embodiment shown in FIG. 11B, a bottom surface 47′ of rubber plug 45′ is generally concave. No engaging groove for fixing a lower fixing piece 48 of the rubber plug 45 is formed to an opening part for connecting 43, while the lower fixing piece 48 of the rubber plug 45 is sandwiched between the inner peripheral surface of the lid member 40 and the outer peripheral surface of the opening part for connecting 43 to be fixed. The other components of the liquid mixing and injecting tool B are the same as those of the liquid mixing and injecting tool A described in above. Therefore the explanation is abbreviated by indicating the same components with the same referential numbers.

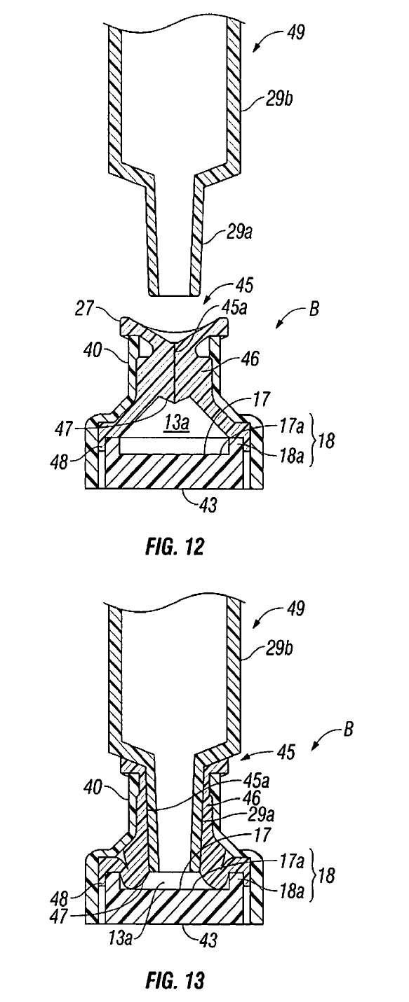

By configuration it in this way, as shown in FIG. 11, when the male luer part 29a of the syringe 49 shown in FIG. 12 is inserted into the rubber plug 45 while the slit 45a is occluded, the male luer part 29a expands the slit 45a as pushing down the rubber plug body 46 into the lid member 40 as downwardly moved. In this time, the rubber plug body 46 extends the upper fixing piece 27 while the body 46 itself is stretched and downwardly moved. Also, the male luer part 29a is in the state as shown in FIG. 13 as inserted into the slit 45a to communicate with the chamber part 11.

In other word, the rubber plug body 46 is also pressed down into the lid member 40 by the male luer part 29a and the lower end portion thereof is contacted to the rubber plug receiving part 18 to avoid the formation of any gap where air is to be accumulated in the tip portion of the male luer part 29a when it is inserted into the slit 45a. Then, the small protrusion 47 of the rubber plug body 46 is expanded by the tip portion of the male luer part 29a as shown in FIG. 13, thereby forming a tapered curved surface 47a downwardly expanded between the outer peripheral tip end of the male luer part 29a and the upper surface 17a of the weir part 17.

By the tapered curved surface 47a, the opportunity of the formation of the gap where air is to be accumulated in the tip portion of the male luer part 29a is further avoided. In this way, since the formation of any spaces where air is to be accumulated within the chamber part 11 can be inhibited by the liquid mixing and injecting tool B, the complicated operation to remove out the air within the chamber part 11 or the generation of bacteria within the chamber part 11 can be avoided. Another effects of the liquid mixing and injecting tool B are the same as those of the liquid mixing and injecting tool A described in above.

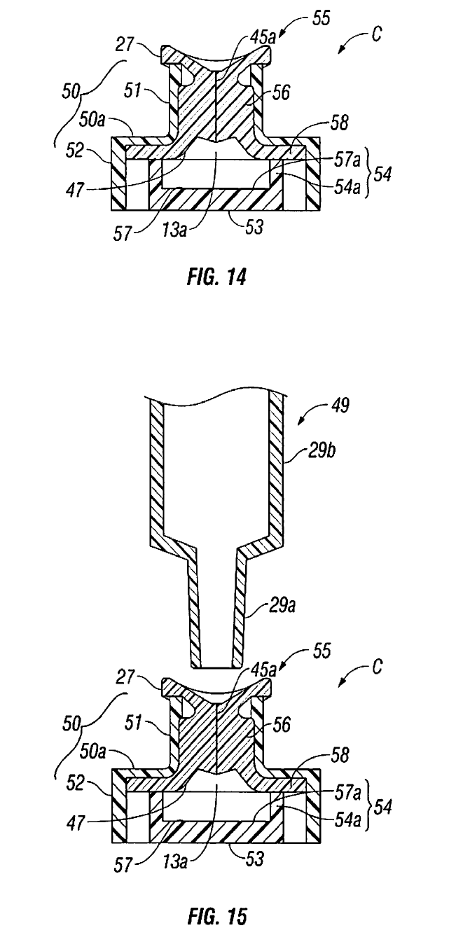

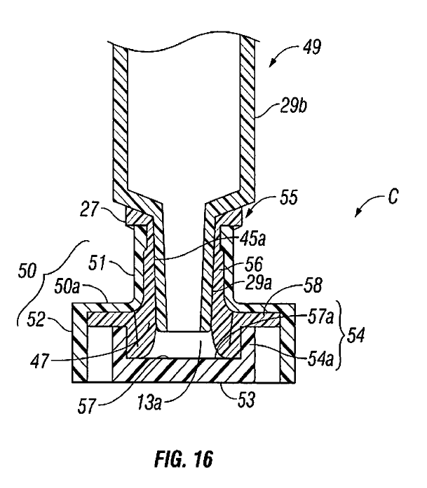

FIGS. 14 to 16 show a liquid mixing and injecting tool C of the modified embodiment of the liquid mixing and injecting tool B, in which the syringe 49 is connected thereto. In the liquid mixing and injecting tool C, the portion in a rubber plug 55 which is sandwiched and fixed between a lid member 50 and an opening part for connecting 53 includes not only a lower fixing piece 58 but also a lower side portion of a rubber plug body 56. Further, a step of great difference rather than a tapered part is provide between the upper part 51 and the lower part 52 of the lid member 50. Also, the tip portion of the lower fixing piece 58 is not downwardly extended along the inner peripheral surface of the lid member 50 and the outer peripheral surface of the opening part for connecting 53 but along the lower surface of a horizontal part 50a formed between the upper portion 51 and the lower portion 52 of the lid member 50 in the horizontal direction to be fixed.

Furthermore, the height between the upper end portion of a side wall 54a comprising a rubber plug receiving part 54 and the upper surface 57a of a weir part 57 is provided so as to be slightly greater than that between the upper portion of the side wall 18a and the upper surface 17a of the weir part 17 in the liquid mixing and injecting tool B. The other components of the liquid mixing and injecting tool C are the same as those of the liquid mixing and injecting tool B described in above. Therefore, the explanation is abbreviated by indicating the same components with the same referential numbers. Similar effects of the liquid mixing and injecting tool B described in above can be obtained by this liquid mixing and injecting tool C.

The liquid mixing and injecting tool in accordance with the present disclosure is not intended to be limited to the embodiments described in above, any modification thereof can be made. For example, while the active plug type including the valve 30 as a liquid mixing and injecting tool is employed in the above embodiments, other type in which the valve is not provided to flow a liquid medicine and the like can be flowed all the time from the upstream branch tube to the downstream branch tube and another liquid medicine can be flowed from the upstream branch tube to the chamber part at the same time can be employed. Alternatively, another type of liquid mixing and injecting tool in which the upstream branch tube is not provided, branch tubes are formed only by the upper branch tube and the downstream branch tube can be employed.

While the width of the partition wall 35 is the same as the total length of the widths of the groove 33 and the circumference groove 34a of the groove 34 and the weir part 17 having the same width of that of the partition wall 35 is provided at the upper portion of the partition wall 35 in the embodiments described in above, such widths of the partition wall 35 or weir part 35 can appropriately be changed. For example, when the width of the partition wall 35 or the weir part 35 is shorter than the total length of widths of the groove 33 and the circumference groove 34a of the groove 34, the flow rate of the liquid can be increased.

We claim:

1. A liquid mixing and injecting device comprising:
a chamber part including:
a plurality of branch tubes which extend from the chamber part in different directions, the plurality of branch tubes including an upper branch tube extending upwardly from the chamber part;
a rubber plug having a slit formed therein, the rubber plug being supported in the upper branch tube in a position to occlude the upper branch tube, the slit being dimensioned to sealingly receive a connecting tube to fluidly couple the connecting tube to the chamber part, the rubber plug including:
a body having a fixing piece secured to the upper branch tube;
wherein the body of the rubber plug is pressed into a lower side of the upper branch tube along an outer peripheral surface of a connecting tube when the connecting tube is inserted into the slit;
a receiving part positioned to receive a lower end part of the rubber plug, the receiving part being configured to control deformation of the lower end part of the rubber plug when the rubber plug is pressed into an inner end of lower side of the upper branch tube to prevent formation of an air gap in a flow path communicating the connecting tube with the chamber part.

2. The liquid mixing and injecting device according to claim 1, wherein the plurality of branch tubes include the upper branch tube, a substantially horizontal upstream branch tube extending from the chamber part, and a substantially horizontal downstream branch tube extending from the chamber part, and a weir part extending into the lower side of the upper branch tube when liquid flows between the upstream branch tube and the downstream branch tube through the chamber part, the receiving part being formed on opposite sides of the weir part.

3. The liquid mixing and injecting device according to claim 2, wherein a lower surface of the body of the rubber plug is generally dome shaped.

4. The liquid mixing and injecting device according to claim 1, wherein the lower end of the body of the rubber plug has a tapered shape which expands outwardly towards the receiving part when the rubber plug is pressed into the lower side of the upper branch tube.

5. The liquid mixing and injecting device according to claim 1, wherein the upper branch tube has a generally cylindrical shape and has a diameter adjacent the chamber part which is greater than a diameter adjacent an opening part of the upper branch tube, wherein the rubber plug expands outwardly when the body of the rubber plug presses against the upper branch tube.

6. The liquid mixing and injecting device according to claim 1, wherein the upper branch tube includes an opening part which is integrally formed with the chamber part and a generally cylindrical member attached to the opening part.

7. The liquid mixing and injecting device according to claim 1, wherein the fixing piece of the rubber plug includes an upper fixing piece which is secured adjacent an upper end of the generally cylindrical member and a lower fixing piece which is secured between the opening part and the generally cylindrical member.

8. The liquid mixing and injecting device according to claim 1, wherein a valve is supported within the chamber part, the valve being movable to fluidly connect any one of the plurality of branch tubes with the chamber part.

9. The liquid mixing and injection device according to claim 1, wherein an inner peripheral surface of the upper branch tube defines a tapered surface which becomes gradually thinner from the chamber part toward an upper end of the upper branch tube.

10. The liquid mixing and injection device according to claim 9, wherein a portion of the rubber plug closely contacting the tapered surface of the upper branch tube becomes gradually thinner from the chamber part towards the upper end of the upper branch tube, wherein when pressure is applied to the rubber plug from the chamber part, the rubber plug is pressed onto the tapered surface of the upper branch tube to maintain the slit of the rubber plug in an obstructed condition.

11. The liquid mixing and injection device according to claim 10, wherein an angle defined between an outer peripheral surface of the rubber plug which is to be positioned adjacent the tapered surface of the upper branch tube and a vertical axis, prior to attachment of the rubber plug to the upper branch tube, is greater than an angle defined between the tapered surface of the upper branch tube and the vertical axis.
